(19) United States
(12) Patent Application Publication
(10) Pub. No.: US 2024/0277290 A1
(43) Pub. Date: Aug. 22, 2024

(54) WEARABLE DIAGNOSTIC DEVICE

(71) Applicant: E-NOVIA S.P.A., Milano (MI) (IT)

(72) Inventors: Davide BRUSCHI, San Giuliano Milanese (MI) (IT); Luca TESTA, Milano (MI) (IT); Nicola Giuseppe TRAPLETTI, Grone (BG) (IT); Glauco BIGINI, Milano (MI) (IT); Eleonora D'ARNESE, Aosta (AO) (IT); Letizia CLEMENTI, Busto Garolfo (MI) (IT); Marco Domenico SANTAMBROGIO, Cinisello Balsamo (MI) (IT)

(73) Assignee: E-NOVIA S.P.A., Milano (MI) (IT)

(21) Appl. No.: 18/293,814

(22) PCT Filed: Aug. 3, 2022
(86) PCT No.: PCT/IB2022/057188

§ 371 Date: Jan. 31, 2024

(30) Foreign Application Priority Data
Aug. 4, 2021 (IT) 102021000021125

(51) Int. Cl. A61B 5/00; A61B 5/26; A61B 5/282; A61B 5/332; A61B 5/339

(52) U.S. Cl. CPC A61B 5/6806; A61B 5/26; A61B 5/282; A61B 5/332; A61B 5/339; A61B 2560/0468

(57) ABSTRACT

A diagnostic device for acquiring a twelve-lead electrocardiogram of a patient is provided. The diagnostic device has a first glove, or right glove, and a second glove, or left glove, configured to be worn in the hands of a user and intended, in use, to be placed at a portion of a patient's chest to acquire an electrocardiogram of the patient. The first glove, or right glove, and the second glove, or left glove, have a plurality of electrodes configured to detect at least three cardiac signals, corresponding to three cardiac leads. The diagnostic device also includes an electronic unit configured to receive and process said at least three cardiac signals to obtain a 12-lead electrocardiogram.

### TECHNICAL FIELD

The present invention refers to a diagnostic device. In more detail, the present invention refers to a diagnostic device for acquiring an electrocardiogram of a patient, preferably under non-hospital conditions, such as in the context of an ambulance rescue, medical guard, or mountain rescue.

The diagnostic device according to the present invention can be worn by a user for the purpose of acquiring a twelve-lead electrocardiogram of a patient.

### STATE OF THE ART

The electrocardiogram is one of the main examinations related to the rhythm and electrical activity of the heart. It proves to be an essential examination nowadays, as heart disease is one of the leading causes of death worldwide.

In many cases, however, this examination is a valuable diagnostic tool for an initial diagnosis only if performed with appropriate instruments. In particular, in order to detect particular pathologies, such as cardiac ischemia, a 12-lead electrocardiogram is required. This type of electrocardiogram typically requires the placement of ten electrodes on a patient at rest and in a controlled environment, using specific and often high-cost machines.

Given the complex conditions required and the technology needed to perform this type of examination, it is therefore apparent that in some situations, such as during an emergency outside a hospital facility, it is often not possible to obtain an accurate and detailed cardiac analysis. In detail, in particular emergency conditions, such as during an ambulance rescue or in emergency medical care, it is often not possible to obtain a detailed and reliable analysis of a patient's cardiac condition.

Nowadays, known electrocardiogram machines generally require the placement of ten electrodes through which are recorded the twelve leads necessary for a detailed electrocardiogram.

In particular, hospital devices for electrocardiogram acquisition are often complicated in use, have low portability features, and require the expertise of a doctor to interpret the obtained results. In addition, such devices are characterized by high costs.

For example, the placement of electrodes in a patient's body requires the experience and the manual skill of an operator.

In order to simplify this issue, some devices are known to simplify the placement of the electrodes themselves in the patient's body. Such devices may include, for example, holders intended to be placed on the patient's body and onto which the electrodes are applied. Like traditional hospital devices, however, they also suffer from issues related to their large volume, mainly due to the large number of electrodes and the resulting large need for memory, computing power, and battery capacity of the device itself. In addition, the large number of electrodes also requires the placement of the electrodes at very different and distant locations in the patient's body, such as in the chest area and at the patient's wrists and ankles. Consequently, known supports for improved electrode placement difficulties result often in large size and weight, resulting in reduced portability and use limited to only the hospital or outpatient setting.

Nowadays, there are also some devices available that are configured for electrocardiogram acquisition under other than hospital conditions. Such devices, while simpler in use and easily portable and/or wearable by a patient or user, do not allow recording of all twelve leads of a detailed electrocardiogram. In fact, many of such devices allow the recording of only one or six leads. Consequently, only normal rhythm, conditions of tachycardia or bradycardia, or atrial fibrillation can be identified through such devices.

Such devices, while advantageous in some respects, produce results characterized by limited clinical value. For example, the pathologies detectable by these portable and/or wearable devices account for only a small portion of the total of major cardiac pathologies, which often result in patient death. In detail, in many cases, major cardiac pathologies are identifiable only by twelve-lead electrocardiograms.

### SUMMARY OF THE INVENTION

The problem underlying the present invention is to overcome the drawbacks of traditional solutions by allowing a detailed twelve-lead electrocardiogram to be obtained through a diagnostic device that is simple to use, has limited cost, and is easily worn and/or carried by a user so that a cardiac analysis of a patient can be obtained even under non-ambulatory conditions.

Thus, the task of a diagnostic device according to the present invention is to solve this problem.

Within that task, one purpose of the invention is to enable the obtaining of a twelve-lead electrocardiogram using a limited number of electrodes.

Within that task, one purpose of the invention is also to obtain a twelve-lead electrocardiogram through a user-wearable device.

Moreover, a purpose of the present invention is to propose a diagnostic device capable of acquiring a twelve-lead electrocardiogram of a patient that is user-friendly.

These tasks, as well as these and other purposes that will better appear below, are achieved by a diagnostic device according to the attached independent claim.

Advantageously, the diagnostic device for acquiring a twelve-lead electrocardiogram of a patient according to the present invention comprises a first glove, or right glove, and a second glove, or left glove which are configured to be worn in the hands of a user and intended, in use, to be placed at a portion of the patient's chest for the purpose of acquiring the electrocardiogram. Consequently, the diagnostic device for acquiring electrocardiogram according to the present invention is highly wearable and easily reusable.

The first glove, or right glove, and the second glove, or left glove, comprise a plurality of electrodes configured to detect at least three cardiac signals, or three cardiac leads. Preferably, the plurality of electrodes is configured to detect three cardiac signals, or leads. Consequently, the number of electrodes required is fewer than in conventional devices for acquiring a twelve-lead electrocardiogram.

Advantageously, the use of wearable gloves by an operator in which a plurality of electrodes aimed at detecting a patient's electrocardiogram are arranged ensures, on the one hand, extreme ease of use, as the operator is required to simply place his or her hands, with the gloves on, in a patient's chest, and on the other hand, a high portability and flexibility of use of the device itself, which, thanks to the miniaturization of all its components, can be simply transported and used not only in hospital or outpatient conditions, but also in different conditions, such as in ambulance rescue, medical guard, mountain rescue, or in environments and territories in which there are generally no hospital facilities.

Advantageously, moreover, the fact that gloves are used to place the electrodes in the patient's body greatly simplifies the operation of acquiring the electrocardiogram by the user and makes such acquisition much faster and more repeatable than with traditionally used devices.

Still, advantageously, the use of gloves comprising electrodes allows the operator to be able to perform various activities and, if necessary, acquire the patient's electrocardiogram. In other words, the arrangement of the electrodes in a pair of gloves worn by an operator allows the detection of an electrocardiogram quickly and easily, if necessary, even if the operator himself has to perform other tasks on the patient.

Furthermore, the diagnostic device according to the present invention includes an electronic unit configured to receive and process the cardiac signals detected by the plurality of electrodes in order to obtain a twelve-lead electrocardiogram. In other words, the electronic unit enables a twelve-lead electrocardiogram to be obtained from only three cardiac signals, or leads.

Detailed features of a diagnostic device according to the invention are given in the dependent claims.

Further features and advantages of the invention will result more from the description of a preferred, but not exclusive, form of embodiment of a safety device and actuator for a fixture comprising the same, according to the invention, illustrated, in a form of embodiment depicted by way of illustration and not limitation in the united tables of drawings below.

### BRIEF DESCRIPTION OF THE DRAWINGS

FIG. 1 shows an actuator for an infix according to the present invention in view of the diagnostic device according to the present invention;

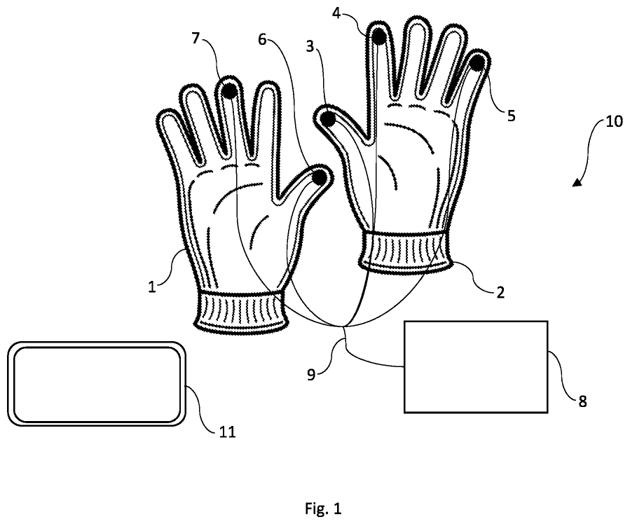

FIG. 2 shows a view of a portion of a patient's chest in which the position, in use, of the electrodes of the diagnostic device according to the present invention is highlighted.

### DETAILED DESCRIPTION

With particular reference to the attached figures, a diagnostic device for acquiring a twelve-lead electrocardiogram of a patient is globally referred to as 10.

In detail, such diagnostic device 10 comprises a first glove 1, or right glove, and a second glove 2, or left glove. Such first glove 1 and second glove 2 are configured, in use, to acquire a plurality of cardiac signals from a patient, each corresponding to a cardiac lead. In detail, the first glove 1, or right glove, and the second glove 2, or left glove, are configured to acquire, or detect, at least three cardiac signals, or cardiac leads, of a patient.

In addition, the diagnostic device 10 includes a processing unit 8, configured to receive the at least three cardiac signals detected by the first glove 1 and the second glove 2 and to process such at least three cardiac signals detected by the first glove 1 and the second glove 2 in order to obtain a twelve-lead electrocardiogram.

Preferably, the processing unit 8, 10 comprises an electronic unit 8, preferably arranged on one of the two gloves, and configured to receive the at least three cardiac signals detected by the first glove 1 and the second glove 2. Such an electronic unit may preferably be operationally associated with a portable device 11 of the processing unit 8, 10, such as a tablet or smartphone or computer, configured to process the three cardiac signals detected by the electrodes.

In more detail, the acquisition of the at least three cardiac signals, preferably the three cardiac signals, or leads, by means of the first glove 1 and the second glove 2 is done through a plurality of electrodes arranged in the same first glove 1 and second glove 2. Specifically, in order to detect the at least three cardiac signals through the plurality of electrodes 3, 4, 5, 6, 7, the first glove 1, or right glove, and the second glove 2, or left glove, are configured, in use to be worn in the hands of an operator or user and intended, in use, to be placed at a portion of a patient's chest in order to acquire the at least three cardiac signals.

Preferably, according to one aspect of the present invention, each electrode of the plurality of electrodes 3, 4, 5, 6, 7 is a contact electrode. Such electrodes, besides being particularly inexpensive, are readily available.

According to a further preferred aspect, each electrode of the plurality of electrodes 3, 4, 5, 6, 7 is a capacitive electrode. Advantageously, such electrode types allow an acquisition of a cardiac signal even through the patient's clothes. Therefore, it follows that the use of this type of electrode further simplifies the acquisition of a patient's electrocardiogram without the need to undress the patient. Advantageously then, the acquisition of a patient's electrocardiogram is also simplified in an emergency situation, such as in an ambulance.

More in detail, according to one aspect of the present invention, each electrode of the plurality of electrodes 3, 4, 5, 6, 7 is placed at a terminal portion of the first glove 1, or right glove, and/or the second glove 2, or left glove. “Terminal portion,” in the context of this invention, means portions of the first glove 1 and/or the second glove 2 such that, when such first glove 1 and/or such second glove 2 is/are worn by an operator, such portions correspond to a terminal portion, or last phalanx of one or more fingers of the hand wearing the corresponding glove. In other words, each electrode 3, 4, 5, 6, 7 is placed in a glove 1, 2, preferably in an outer surface of a glove 1, 2, even more preferably at a portion of a glove 1, 2 itself configured to cover a fingertip portion of a finger of a hand of a user.

According to an advantageous aspect of the present invention, the first glove 1, or right glove, and the second glove 2, or left glove, comprise a total of five electrodes 3, 4, 5, 6, 7. In other words, the sum of the electrodes arranged on the first glove 1, or right glove, and the second glove 2, or left glove, is five.

It advantageously follows that the use of the diagnostic device according to the present invention allows the acquisition of a twelve-lead electrocardiogram based on only three cardiac signals, or leads. Consequently, the number of electrodes required for the acquisition of such an electrocardiogram is advantageously limited compared to devices of known art. The limited number of electrodes required for the acquisition of the twelve-lead electrocardiogram significantly simplifies and speeds up the operations required to obtain the same.

As previously anticipated, to detect the at least three cardiac signals for the acquisition of the twelve-lead electrocardiogram, the first glove 1, or right glove, and the second glove 2, or left glove, are configured, in use to be worn in the hands of a user and intended, in use, to be placed at a portion of a patient's chest in order to acquire the at least three cardiac signals. Specifically, the first glove 1, or right glove, is configured to be worn in a user's right hand, while the second glove 2, or left glove, is configured to be worn in a user's left hand for the purpose of acquiring a patient's electrocardiogram.

Moreover, in use, in order to detect the at least three required cardiac signals, the first glove 1, or right glove, is intended to be placed in a right-upper portion of a patient's chest, while the second glove 2, or right glove, is intended to be placed in a left-lower portion of a patient's chest. Advantageously, the fact that electrodes 3, 4, 5, 6, 7 are arranged in a user-wearable device, and in particular in a pair of gloves that can be worn by a user himself, together with the fact that these electrodes 3, 4, 5, 6, 7 are arranged at terminal portions of the gloves themselves and that these are intended, in use, to be placed at a region of the patient's chest, greatly simplifies the operations necessary for the acquisition of the electrocardiogram. In fact, not only the limited number of electrodes, but also the placement of the electrodes in the patient's body, makes the acquisition of the twelve-lead electrocardiogram much easier, quicker, and possible even in non-ambulatory conditions. In detail, the placement of the electrodes at terminal portions of the first glove 1, or right glove, and the second glove 2, or left glove, requires, in order to acquire the at least three necessary cardiac signals, the simple placement of the user's hands at the patient's chest.

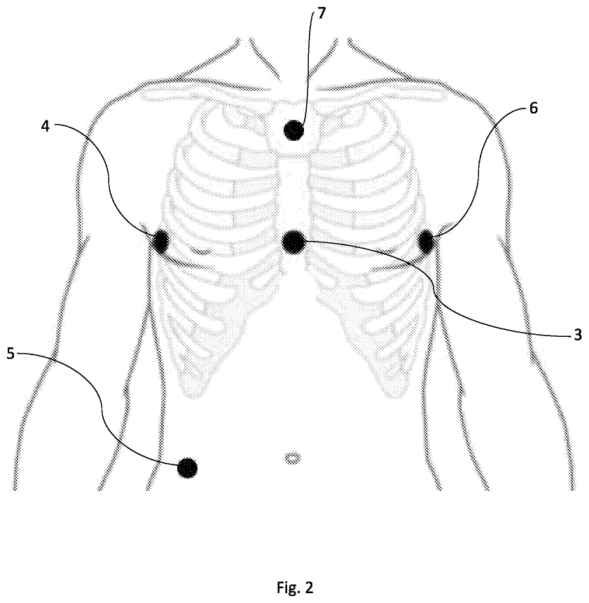

With particular reference to FIG. 2, in detail, in use, a first electrode 3 is intended to be placed at a lower part of a patient's sternum, specifically at the level of the V intercostal space. According to this aspect, a second electrode 4 is intended, in use, to be placed at a right mid-axillary line, at the same level, or substantially at the same level as the first electrode 3. A third electrode 5 is intended, in use, to be preferably placed on a right side of a patient, preferably under the patient's sixth rib. A fourth electrode 6 is intended, in use to be placed on a left middle axillary line, at the same level or substantially the same level as the first electrode 3. Finally, a fifth electrode 7 is intended, in use, to be placed on an upper part of a patient's sternum.

Such placement of the five electrodes 3, 4, 5, 6, 7 corresponds to the electrode placement provided by the EASI™ method (https://www.theonlinelearningcenter.com/Assets/PMDCBT/PIIC_Fundamentals_1.0/shell/viewer/swfs/assets/downloads/easi.pdf), which involves detecting three cardiac signals, or three leads, from five electrodes placed as described above and obtaining the additional nine leads required for a 12-lead electrocardiogram mathematically from the three signals detected by the electrodes. Advantageously, therefore, the EASI™ method enables the obtaining of a twelve-lead electrocardiogram with the use of a limited number of electrodes compared with traditional hospital techniques used for obtaining a qualitatively comparable electrocardiogram. In addition, the electrodes provided by the EASI™ method are placed in a limited portion of the patient's body, without the need to place electrodes at the patient's wrists and ankles, as is the case with traditional twelve-lead electrocardiograms, for example. The limited portion of the patient's body in which the electrodes are to be placed according to the EASI™ method allows the use of a holder, such as a pair of gloves, for simple and quick placement of the electrodes.

In other words, the combination of the use of gloves comprising electrodes and the use of the EASI™ method allows the calculation of a twelve-lead electrocardiogram using a limited number of electrodes and, thanks to a support such as gloves, allows simple placement of the electrodes themselves in a patient's body.

Ultimately, therefore, the arrangement of the five electrodes 3, 4, 5, 6, 7 at a pair of gloves worn by a user greatly simplifies the placement of the electrodes themselves at the aforementioned specific points on the patient's chest. In other words, in order to acquire an electrocardiogram of a patient, it is sufficient, according to the present invention, to arrange the open hands of a user wearing the diagnostic device object of the present invention at a right-superior area of a patient's chest and left-inferior area of the patient's chest. The size of the user's hands, and therefore of the first glove 1, or right glove, and the second glove 2, or left glove, together with the fact that the five electrodes are placed at terminal portions of the gloves themselves, will allow for very simple, accurate and rapid placement of the electrodes at the locations provided by the EASI™ method.

According to a preferred aspect of the present invention, the first glove 1, or right glove, includes two electrodes 6, 7, while the second glove, or left glove, includes three electrodes, 3, 4, 5. Preferably, the two electrodes 6, 7 of the first glove 1, or right glove, are placed at two distinct terminal portions of the first glove 1, or right glove, itself, while the three electrodes 3, 4, 5 of the second glove 2, or left glove, are placed at three distinct terminal portions of the second glove 2, or left glove.

The choice of using five electrodes arranged in the gloves ensures, on the one hand, an obtaining of an electrocardiogram according to the EASI™ method, and on the other hand greatly simplifies the electronics required for processing the signals obtained for the purpose of calculating the twelve-lead electrocardiogram. The number of electrodes may possibly vary, for example by providing for the elimination of a ground electrode, but the arrangement of five electrodes in the two gloves is particularly advantageous for obtaining a good quality electrocardiogram and for the miniaturization and ease of use of the device itself.

Even more specifically, according to a preferred aspect of the present invention, the first electrode 3 is placed at an end portion of the second glove 2, or left glove, intended to cover a thumb finger of the wearing operator, while a second electrode 4 is placed at an end portion of the second glove 2, or left glove, intended to cover an index or middle finger of the wearing operator, and preferably intended to cover the index finger of the wearing operator. A third electrode 5 is placed at an end portion of the second glove 2, or left glove, intended to cover the little finger, or ring finger, preferably the little finger, of the wearing operator. Furthermore, according to this aspect, a fourth electrode 6 is placed at an end portion of the first glove 1, or right glove, intended to cover a thumb finger of the wearing operator, while a fifth electrode 7 is placed at an end portion of the first glove 1, or right glove, intended to cover an additional finger, preferably the middle finger, of the operator wearing it.

Such electrode placement ensures the best ease and facility of electrode placement on a patient's body by an operator. In other words, the specific arrangement of the electrodes at the end portions of the gloves worn by the operator facilitates the operator in placing the electrodes at the correct locations on the patient's body, allowing for simplified, rapid, and easily repeatable electrocardiogram detection.

Such arrangement of electrodes 3, 4, 5, 6, 7 allows easy placement of the same in relation to the points provided by the EASY™ method. In fact, in order to detect the three cardiac signals, or leads, necessary for the acquisition of the twelve-lead electrocardiogram, it is sufficient for an operator or user to correctly arrange the hands, with their gloves, in an open position, with the fingertips and consequently the terminal portions of the gloves where electrodes 3, 4, 5, 6, 7 are arranged facing the patient's body.

Furthermore, advantageously, the use of two separate gloves, each preferably including a plurality of electrodes, provides a high degree of flexibility in use, resulting in the possibility of correctly positioning a first set of electrodes and a second set of electrodes independently of each other.

According to a preferred aspect of the present invention, as previously anticipated, the diagnostic device 10 is configured to detect at least three cardiac signals, or three leads, via electrodes 3, 4, 5, 6, 7. In detail, a pair of electrodes allows the detection of an electrical potential difference, or lead, between the two points where the electrodes are placed.

Specifically, in the case of the diagnostic device 10 according to the present invention, a first cardiac signal, or first lead, is detected between the fourth electrode 6 and the fifth electrode 7. In other words, a first cardiac signal is detected between an electrode placed at a terminal portion of the thumb finger of the first glove 1, or right glove, and an electrode preferably placed at the middle finger of the first glove 1, or right glove. In still other words, the first signal is detected between an electrode placed, in use, on a left middle axillary line and an electrode placed, in use, on an upper portion of a patient's sternum.

According to this aspect, a second cardiac signal, or second lead, is detected between the fourth electrode 6 and the second electrode 4. In other words, the second cardiac signal is detected between an electrode placed at a terminal portion of the thumb finger of the first glove 1, or right glove, and an electrode placed preferably at the index finger of the second glove 2, or left glove. In still other words, a second cardiac signal is detected between an electrode placed, in use at a left middle axillary line and an electrode placed, in use at a patient's right middle axillary line.

Also according to this preferred aspect, a third cardiac signal, or third lead, is detected between the first electrode 3 and the fifth electrode 7. In other words, the third cardiac signal is detected between an electrode placed at a terminal portion of a thumb finger of the second glove 2, or left glove and an electrode placed preferentially at a middle finger of the first glove 1, or right glove. In still other words, the third cardiac signal is detected between an electrode placed, in use, at a lower portion of a patient's sternum, particularly at the level of the V intercostal space and an electrode placed, in use, at an upper portion of a patient's sternum.

Advantageously then, by means of the five electrodes 3, 4, 5, 6, 7 placed at terminal portions of the first glove 1 and the second glove 2 worn by an operator or user and appropriately placed in a chest region of a patient, it is possible to detect the three cardiac signals, or leads, provided by the EASI™ method in order to acquire a twelve-lead electrocardiogram of a patient.

In order to detect said three cardiac signals, or three leads, the first glove 1, or right glove, and the second glove 2, or left glove, are connected to each other and to the electronic unit 8 through a shielded cable 9. In more detail, each electrode 3, 4, 5, 6, 7 is connected to the electronic unit 8 through a shielded cable 9. The use of shielded cables allows the detection of cardiac signals and their transfer to the electronic unit 8 preventing the occurrence of electromagnetic fields and noise that could adversely affect the obtained electrocardiogram.

In combination or as an alternative, wireless communication means configured to transfer the signals detected via electrodes 3, 4, 5, 6, 7 to electronic unit 8 can be provided.

In addition, in combination with or as an alternative to the shielded cable 9 or wireless means of communication, printed electronics, such as printed circuit boards, e.g. arranged on the gloves 1, 2, may be provided for connecting the electrodes 3, 4, 5, 6, 7 and the electronic unit 8. For example, electrodes in one glove may be connected to each other via a printed circuit board. In addition, electronic unit 8 can also be connected to the electrodes in a glove via a printed circuit board. Eventually, the two gloves can be connected to each other via a shielded cable 9. This solution, advantageously, allows for a significant reduction in space and elimination of wiring with obvious benefits on the use of the diagnostic device 10.

The electronic unit 8 is configured to receive and possibly process the biometric signals detected by the electrodes 3, 4, 5, 6, 7.

Preferably, the electronic unit 8, in use, is connected to both gloves 1 and 2 and arranged on the back of one between the first glove 1 and the second glove 2.

Electronic unit 8 may include an on/off button for the diagnostic device 10. In addition, the electronic unit 8 may include an input port for connection with the first glove 1 and/or the second glove 2 via, preferably, a shielded cable 9.

Also, preferably, the electronic unit 8 may include a battery to power the diagnostic device 10. Preferably, such a battery is a rechargeable battery.

The diagnostic device 10 may further comprise a portable device 11, such as a tablet or smartphone or computer, operatively connected to the electronic unit 8.

Preferably, the portable device 11 is configured to process the at least three signals detected via the electrodes 3, 4, 5, 6, 7 and to graphically represent the acquired twelve-lead electrocardiogram.

In other words, the portable device 11 can be configured both to process the cardiac signals transmitted from the electrodes to electronic unit 8, for example, in case the latter was not configured to process the three signals themselves in order to obtain the twelve-lead electrocardiogram, and to graphically display the processed electrocardiogram.

Advantageously, a portable device 11 configured for the processing of the three cardiac signals detected by the electrodes allows for further miniaturization of the diagnostic device 10, particularly relative to the electronics arranged on the gloves, especially relative to the electronic unit 8 which is preferably arranged on one of the two gloves, thus allowing for further portability and versatility of use of the diagnostic device 10 itself. Generally in fact, commonly used portable devices 11, such as smartphones or tablets, are characterized by high computing power and still sufficient for data processing according to the EASI™ method, advantageously decreasing the complexity of the remaining components, especially the electronic unit 8, of the diagnostic device 10. Advantageously also, the portable device 11 can be configured, for possible future development, for example of applications or programs aimed at the detection of particular cardiac conditions on the basis of the detected electrocardiogram, without the eventual need to modify the electronics of the diagnostic device 10, especially of the electronic unit 8.

In detail, the portable device 11 can be configured to compare such signals in order to obtain the three leads, or the three cardiac signals, directly detectable by electrodes 3, 4, 5, 6, 7 according to the EASI™ method. In addition, the portable device 11 is configured to mathematically process those three cardiac signals in order to mathematically obtain the remaining nine leads of a 12-lead electrocardiogram. As such, the portable device 11 may preferably comprise a microprocessor for receiving, noise filtering, and processing the at least three cardiac signals detected via electrodes 3, 4, 5, 6, 7. In other words, the portable device 11 may comprise an algorithm configured for transforming the three leads directly obtainable via the five electrodes 3, 4, 5, 6, 7 into twelve leads as provided by the EASI™ method.

In this regard, preferably, the signal related to the three leads directly obtainable from electrodes 3, 4, 5, 6, 7 can first be filtered, for example, through a low-pass filter for the elimination of possible noise. The signal thus obtained can be further filtered, preferably through a notch filter, to eliminate or reduce any network interference. Then the signal is linearly combined with estimated EASI parameters in order to obtain a 12-lead electrocardiogram. These operations are preferably performed by the portable device 11. Alternatively, these operations can be performed by the electronic unit 8.

Preferably also, the portable device 11 may be configured, preferably via a microprocessor, to analyze the twelve-lead electrocardiogram and to provide a diagnosis of the patient's cardiac condition. For example, parameters related to the number of heartbeats, detection of cardiac conditions such as tachycardia, bradycardia, normal rhythm, or detection of cardiac pathology such as atrial fibrillation can be provided. Alternatively, such operations can be carried out by electronic unit 8.

In addition, the diagnostic device 10 may also include additional sensors to detect when electrodes 3, 4, 5, 6, 7 are placed on the patient. Such information may preferably be visible in the portable device 11.

Said portable device 11 and the electronic unit 8 may preferably be operationally connected via a wireless data transmission protocol for example via the Bluetooth® standard, such that a user may view and analyze the tracing of the twelve-lead electrocardiogram and/or the analysis performed via the portable device 11.

More preferably, the portable device and the electronic unit can be operationally connected via Bluetooth Low Energy (BLE).

The invention thus conceived is susceptible to numerous modifications and variations, all within the scope of protection of the appended claims.

In addition, all details may be replaced by other technically equivalent elements.

Where the operating characteristics and techniques mentioned are followed by reference marks or numbers, such reference marks or numbers have been affixed for the sole purpose of increasing the intelligibility of the description and claims themselves and, accordingly, they do not in any way constitute a limitation on the interpretation of each element identified, by way of example only, by such reference marks or numbers.

1. A diagnostic device for acquiring a twelve-lead electrocardiogram of a patient, wherein said diagnostic device comprises a first glove, or right glove, and a second glove, or left glove, wherein said first glove, or right glove, and said second glove, or left glove, are configured to be worn in the hands of an operator, or user, and intended, in use, to be placed at a portion of a patient's chest to acquire an electrocardiogram of said patient, said first glove, or right glove and said second glove, or left glove, comprising a plurality of electrodes configured to detect at least three cardiac signals, corresponding to three cardiac leads, wherein each electrode of said plurality of electrodes is disposed at an end portion of said first glove, or right glove, and/or of said second glove or left glove, the diagnostic device further comprising a processing unit configured to receive and process said at least three cardiac signals to obtain a twelve-lead electrocardiogram, wherein said first glove, or right glove, and said second glove (2), or left glove, comprise a total of five electrodes.

2. The diagnostic device according claim 1, wherein, in use, a first electrode is intended to be placed at the lower part of a sternum of a patient at the level of the V intercostal space, wherein a second electrode is intended, in use, to be placed at a right mid axillary line at the same level as the first electrode (3), wherein a third electrode is intended, in use, to be optionally placed on a right flank of a patient, below the sixth rib of said patient, wherein a fourth electrode is intended to be placed, in use, on a left mid axillary line at the same level as the first electrode, and wherein a fifth electrode is intended to be placed, in use, on an upper portion of a sternum of a patient.

3. The diagnostic device according to claim 1, wherein said first glove, or right glove, comprises two electrodes and wherein said second glove, or left glove, comprises three electrodes.

4. The diagnostic device according to claim 3, wherein said first electrode is placed at an end portion of said second glove, or left glove, intended to cover a thumb finger of an operator wearing it, wherein said second electrode is placed at an end portion of said second glove, or left glove, intended to cover an index finger or middle finger of an operator wearing it, wherein said third electrode is placed at an end portion of said second glove, or left glove, intended to cover a little finger or ring finger of a an operator wearing it, wherein said fourth electrode is placed at an end portion of said first glove, or right glove, intended to cover a thumb finger of an operator wearing it, and wherein said fifth electrode is placed at an end portion of said first glove, or right glove, intended to cover a further finger of an operator wearing it.

5. The diagnostic device according to claim 2, wherein a first cardiac signal of said at least three cardiac signals is detected between said fourth electrode and said fifth electrode, wherein a second cardiac signal of said at least three cardiac signals is detected between said fourth electrode and said second electrode, and wherein a third cardiac signal of said at least three cardiac signals is detected between said first electrode and said fifth electrode.

6. The diagnostic device according to claim 1, wherein said electrodes are contact electrodes.

7. The diagnostic device according to claim 1, wherein said electrodes are capacitive electrodes.

8. The diagnostic device according to claim 1, wherein the processing unit comprises an electronic unit configured to receive said at least three cardiac signals detected by said plurality of electrodes and a portable device operatively associated with said electronic unit configured to process said at least three cardiac signals to obtain a twelve-lead electrocardiogram.

9. The diagnostic device according to claim 8, wherein said first glove, or right glove, and said second glove, or left glove, are connected to each other and to the electronic unit through a shielded cable.

10. The diagnostic device according to claim 8, wherein said plurality of electrodes are connected to each other and to said electronic unit through a printed circuit board.

11. The diagnostic device according to claim 8, wherein the portable device is further configured to graphically represent the acquired twelve-lead electrocardiogram.
